(19) United States
(12) Patent Application Publication
(10) Pub. No.: US 2022/0301164 A1
(43) Pub. Date: Sep. 22, 2022

(54) CULTURE MANAGEMENT APPARATUS, ESTIMATION METHOD, AND COMPUTER-READABLE MEDIUM

(71) Applicant: OLYMPUS CORPORATION, Tokyo (JP)

(72) Inventors: Yoko KAMATO, Tokyo (JP); Elena SHIMOJI, Tokyo (JP); Takuma DEZAWA, Tokyo (JP)

(73) Assignee: OLYMPUS CORPORATION, Tokyo (JP)

(21) Appl. No.: 17/675,180

(22) Filed: Feb. 18, 2022

(30) Foreign Application Priority Data
Mar. 17, 2021 (JP) 2021-043392

(51) Int. Cl. G06T 7/00; C12M 1/36; C12M 1/24; C12M 1/06

(52) U.S. Cl. CPC G06T 7/0014; C12M 41/48; C12M 23/08; C12M 27/02; G06T 2207/30024

(57) ABSTRACT

A culture management apparatus includes a control device that estimates a state of a specific medium component constituting a culture medium based on images including both the culture medium and a cell in the culture medium acquired by an imaging device at different times.

### CROSS REFERENCE TO RELATED APPLICATIONS

This application is based upon and claims the benefit of priority of the prior Japanese Patent Application No. 2021-043392, filed Mar. 17, 2021, the entire contents of which are incorporated herein by reference.

### BACKGROUND OF THE INVENTION

### Field of the Invention

The present disclosure relates to a culture management apparatus, an estimation method, and a computer-readable medium.

### Description of the Related Art

In cell culture, a culture medium is a source of nutrients necessary for a growth of cells. Appropriate management of the culture medium is important for achieving a preferred growth rate of the cells. For example, under a continuous culture method, medium replacement is performed in order to avoid shortage of nutrients and excessive accumulation of metabolite in the culture medium. In addition, under the fed-batch culture method, in order to control the concentration of a specific medium component (substrate), a specific medium component is added to the culture medium during a culture period.

Such a technique related to culture medium management is described in, for example, JP 2012-170366 A. JP 2012-170366 A describes that the culture medium is sampled from a culture tank in the fed-batch culture, and an addition amount of an added culture medium necessary until the next sampling is calculated and determined based on an analysis result of the sampled culture medium.

### SUMMARY OF THE INVENTION

A culture management apparatus according to an aspect of the present disclosure includes a control device. The control device estimates a state of a specific medium component constituting a culture medium based on images. The images include both the culture medium and cells in the culture medium acquired at different times.

An estimation method according to an aspect of the present disclosure includes estimating the state of the specific medium component constituting the culture medium based on the images. The images include both the culture medium and cells in the culture medium photographed at different times.

A computer-readable medium according to an aspect of the present disclosure is a non-transitory computer-readable medium. The non-transitory computer-readable medium stores a program for causing a computer to execute a process of estimating the state of the specific medium component constituting the culture medium on the basis of the images. The images include both the culture medium and cells in the culture medium photographed at different times.

### BRIEF DESCRIPTION OF THE DRAWINGS

FIG. 1 is a schematic configuration diagram illustrating an overall configuration of a culture management apparatus 1 according to a first embodiment;

FIG. 2 is a diagram illustrating a configuration of a culture apparatus 100 according to the first embodiment;

FIG. 3 is a diagram illustrating a configuration of a control device 10 according to the first embodiment;

FIG. 4 is a flowchart illustrating an example of a process performed by the culture management apparatus 1 according to the first embodiment;

FIG. 5 is a flowchart illustrating an example of a state estimation process;

FIG. 6 is a diagram illustrating a cell change information;

FIG. 7 is a diagram illustrating a learned model;

FIG. 8 is a flowchart illustrating an example of a culture environment control process;

FIG. 9 is a diagram illustrating an example of a concentration change of a specific medium component;

FIG. 10 is a flowchart illustrating another example of the state estimation process;

FIG. 11 is a flowchart illustrating still another example of the state estimation process;

FIG. 12 is a flowchart illustrating still another example of the state estimation process;

FIG. 13 is a flowchart illustrating another example of the culture environment control process;

FIG. 14 is a diagram illustrating another example of the concentration change of the specific medium component;

FIG. 15 is a flowchart illustrating an example of a process performed by a culture management apparatus according to a second embodiment;

FIG. 16 is a diagram illustrating an example of a screen displayed on a display device;

FIG. 17 is a flowchart illustrating an example of a process performed by a culture management apparatus according to a third embodiment;

FIG. 18 is a diagram illustrating another example of the screen displayed on the display device;

FIG. 19 is a flowchart illustrating an example of a process performed by a culture management apparatus according to a fourth embodiment;

FIG. 20 is a diagram illustrating a configuration of a culture apparatus 200 according to a fifth embodiment;

FIG. 21 is a diagram illustrating a configuration of a culture apparatus 300 according to a sixth embodiment; and

FIG. 22 is a diagram illustrating a configuration of a culture apparatus 400 including a microscope.

### DESCRIPTION OF THE EMBODIMENTS

Analysis of a sampled culture medium takes time as described in JP 2012-170366 A. Therefore, when a technique of JP 2012-170366 A is used, it is not possible to immediately respond to a concentration change of a medium component to be controlled, and as a result, it is difficult to maintain an appropriate culture environment during a culture period.

Hereinafter, embodiments of the present invention will be described.

### First Embodiment

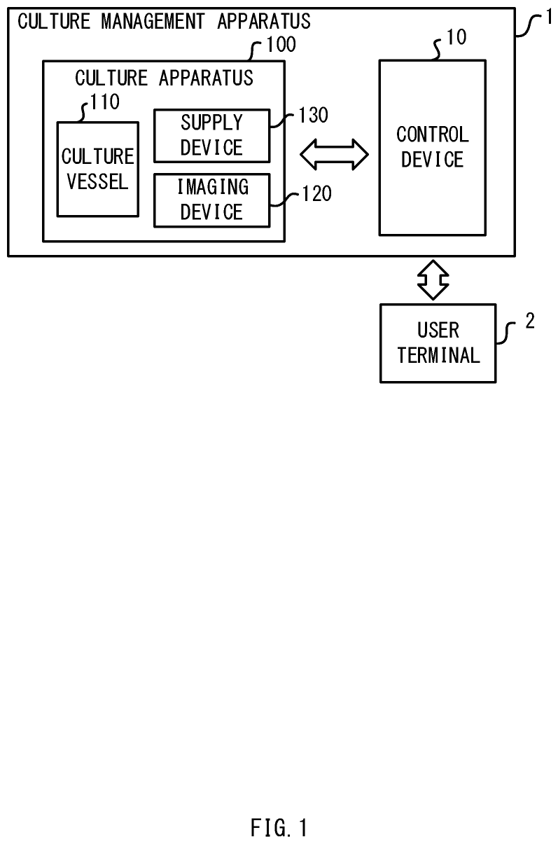

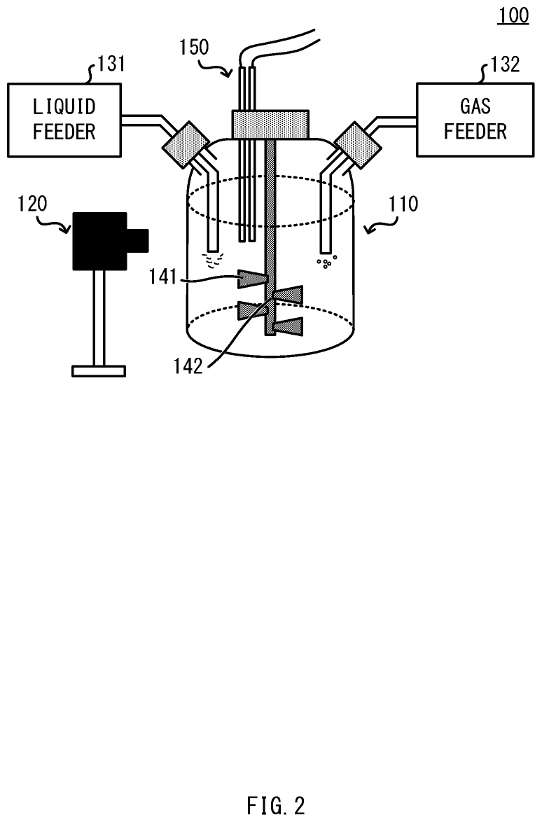

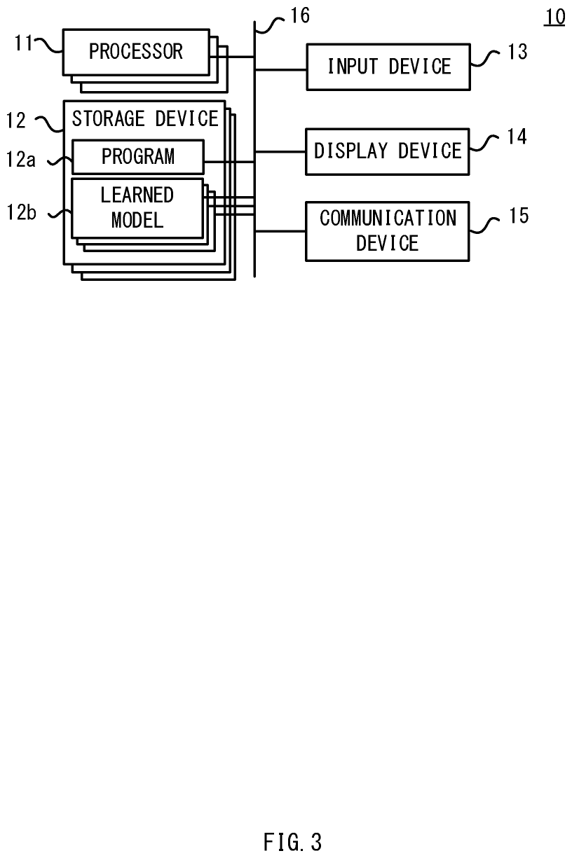

FIG. 1 is a schematic configuration diagram illustrating an overall configuration of a culture management apparatus 1 according to the present embodiment. FIG. 2 is a diagram illustrating a configuration of a culture apparatus 100 according to the present embodiment. FIG. 3 is a diagram illustrating a configuration of a control device 10 according to the present embodiment. Hereinafter, the configuration of the culture management apparatus 1 according to the present embodiment will be described with reference to FIGS. 1 to 3.

The culture management apparatus 1 is an apparatus for efficiently culturing cells by managing a culture environment using fed-batch culture. A method for culturing cells is not particularly limited, but in the present embodiment, suspension culture will be described as an example. The culture management apparatus 1 according to the present embodiment may be used for other culture methods such as carrier suspension culture and static culture (monolayer culture).

As illustrated in FIG. 1, the culture management apparatus 1 includes the culture apparatus 100 that cultures cells and the control device 10 that controls the culture apparatus 100. The culture management apparatus 1 may communicate with a user terminal 2 or may manage the culture environment according to an instruction from the user terminal 2. Further, the culture management apparatus 1 may notify the user terminal 2 of the culture environment in the culture apparatus 100. The user terminal 2 may be connected to the culture management apparatus 1 in a wired manner or may be connected to the culture management apparatus 1 in a wireless manner. The user terminal 2 may be a personal computer (PC), a tablet, a smartphone, or the like. The user terminal 2 may be configured integrally with the culture management apparatus 1.

The culture apparatus 100 is a culture apparatus that performs the fed-batch culture under the control of the control device 10. As illustrated in FIGS. 1 and 2, the culture apparatus 100 includes a culture vessel 110, an imaging device 120, and a supply device 130 (liquid feeder 131 and gas feeder 132).

As illustrated in FIG. 2, the culture apparatus 100 may further include a structure (a stirring blade 141, a rotation shaft 142, a drive unit such as a motor (not illustrated) that drives these components) in which cells are suspended in the culture vessel 110, and various sensors 150 that detect online a temperature, pH, oxygen concentration, carbon dioxide concentration, and the like of the culture medium.

The culture vessel 110 contains a liquid culture medium and cells. When the rotation shaft 142 rotates, a stirring blade 141 fixed to the rotation shaft 142 stirs the liquid culture medium, so that the cells in the culture vessel 110 are cultured in a suspended state in the liquid culture medium.

The imaging device 120 is a device that repeatedly captures an image inside the culture vessel 110 from an outside of the culture vessel 110 and acquires an image including the liquid culture medium and cells suspended in the liquid culture medium. Here, the image including “A” means an image in which “A” is captured. For example, the image including the culture medium and the cells means an image in which both the culture medium and the cells are captured. Note that the cells captured in the image may be a single cell or may constitute a cell mass, a colony, an organoid, a spheroid, a cell sheet, or the like.

The imaging device 120 includes an imaging element. The imaging device 120 is, for example, a digital video camera, but may be a digital still camera. Furthermore, the imaging device 120 may be a camera attached to a microscope. In this case, a sample containing the cell sampled from the inside of the culture vessel 110 may be photographed by the camera attached to the microscope. Images captured by the imaging device 120 at different times is output to the control device 10. The images may be moving images configured with a plurality of frames captured by the digital video camera, or may be a plurality of still images captured by the digital still camera.

The supply device 130 is a device that supplies a predetermined substance to the culture vessel 110 in order to manage the culture environment. The predetermined substance may contain the specific medium component to be controlled in the fed-batch culture or may not contain the specific medium component. The control device 10 controls a feed amount and a feed timing of the predetermined substance supplied from the supply device 130 to the culture vessel 110.

The liquid feeder 131 is a device that feeds a liquid culture medium (hereinafter, the culture medium is referred to as a feed medium as necessary in order to distinguish it from an initial medium in the culture vessel 110 at the start of culture) to be added during a culture period to the culture vessel 110. The feed medium may contain the specific medium component to be controlled in the feed culture. The specific medium component is, for example, a component related to cell metabolism, and is not particularly limited, but may be, for example, glucose, glutamine, or the like. The feed medium may contain a pH adjusting agent or the like. The gas feeder 132 is a device that supplies feed gas to the culture vessel 110. The feed gas may contain, for example, oxygen.

The control device 10 estimates the state of the specific medium component constituting the culture medium in order to appropriately perform the feed culture. Specifically, the control device 10 estimates a state of the specific medium component to be controlled in the feed culture based on at least one of images captured by the imaging device 120. The control device 10 may estimate the state of the specific medium component using information obtained from the sensor 150 in addition to the images.

Note that the images used for estimating the state of the specific medium component desirably includes both the culture medium and the cell. The specific medium component to be estimated may be one or more control targets in the feed culture. In other words, the control device 10 may not necessarily estimate all the control targets on the basis of the image. For example, when the control target in the fed-batch culture includes pH, the control device 10 may control the supply device 130 based on the pH measured by the sensor 150, and as a result, the supply device 130 may supply the pH adjusting agent to the culture vessel 110.

Furthermore, the control device 10 controls the culture apparatus 100 based on the estimated state of the specific medium component. Specifically, the control device 10 may determine the feed amount of the predetermined substance supplied from the supply device 130 to the culture vessel 110 based on the estimated state of the specific medium component. For example, the control device 10 may determine the feed amount of the predetermined substance so as to suppress a change in the state of the specific medium component, and control the supply device 130 to supply the predetermined substance for a determined feed amount. More specifically, for example, the control device 10 may determine the feed amount of the predetermined substance so that a concentration of the specific medium component falls within a certain range, and control the supply device 130 to feed the predetermined substance for the feed amount determined.

Note that the control device 10 may include one or more processors and one or more non-transitory computer-readable media. More specifically, for example, as illustrated in FIG. 3, the control device 10 may include one or more processors 11, one or more storage devices 12, an input device 13, a display device 14, and a communication device 15, and these components may be connected via a bus 16.

Each of the one or more processors 11 is hardware including, for example, a central processing unit (CPU), a graphics processing unit (GPU), a digital signal processor (DSP), and the like, and executes a program 12a stored in one or more storage devices 12 to perform a programmed process. Note that the programmed process may include an estimation process using a learned model 12b stored in the one or more storage devices 12. In addition, the one or more processors 11 may include an application specific integrated circuit (ASIC), a field-programmable gate array (FPGA), and the like.

Each of the one or more storage devices 12 is a non-transitory computer-readable medium and includes, for example, one or more semiconductor memories, and may further include one or more storage devices. The semiconductor memory includes, for example, a volatile memory such as a random access memory (RAM), and a nonvolatile memory such as a read only memory (ROM), a programmable ROM, and a flash memory. The RAM may include, for example, a dynamic random access memory (DRAM), a static random access memory (SRAM), and the like. Other storage devices may include, for example, a magnetic storage device including a magnetic disk, an optical storage device including an optical disk, and the like.

The input device 13 is a device directly operated by a user, and is, for example, a keyboard, a mouse, a touch panel, or the like. The display device 14 is, for example, a liquid crystal display, an organic EL display, a cathode ray tube (CRT) display, or the like. A touch panel may be built in the display. The communication device 15 may be a wired communication module or a wireless communication module.

Note that the configuration illustrated in FIG. 3 is an example of a hardware configuration of the control device 10, and the control device 10 is not limited to this configuration. The control device 10 may be a general-purpose device or a dedicated device. In addition, the control device 10 may not include the input device 13 and the display device 14. And input to and output from the culture management apparatus 1 may be performed by the user terminal 2 including the input device and the display device instead of the control device 10.

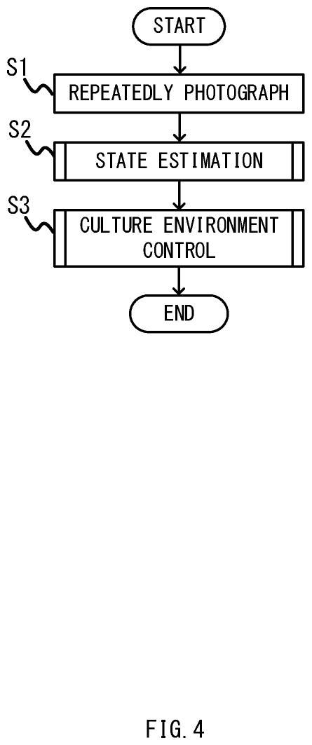

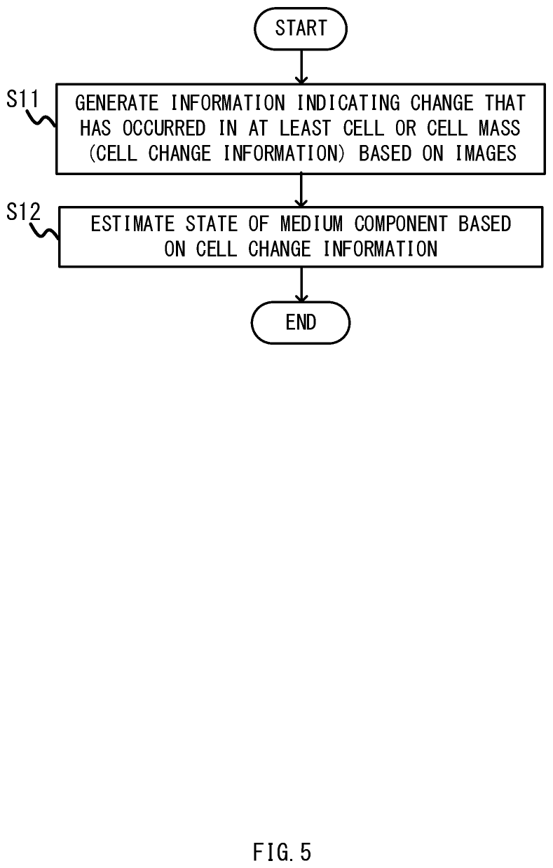

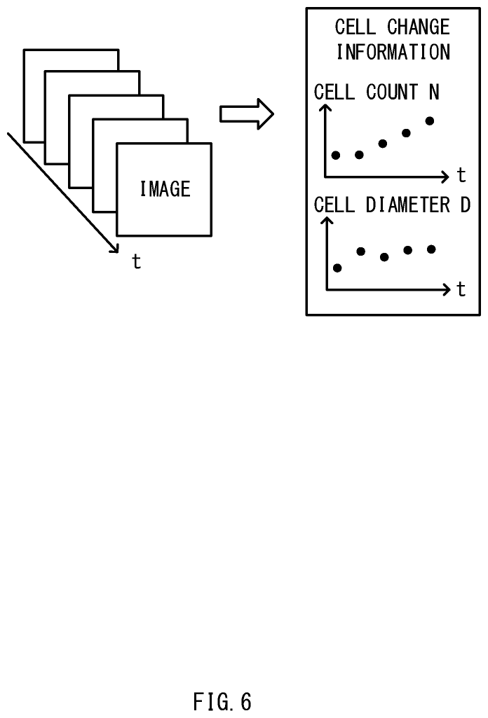

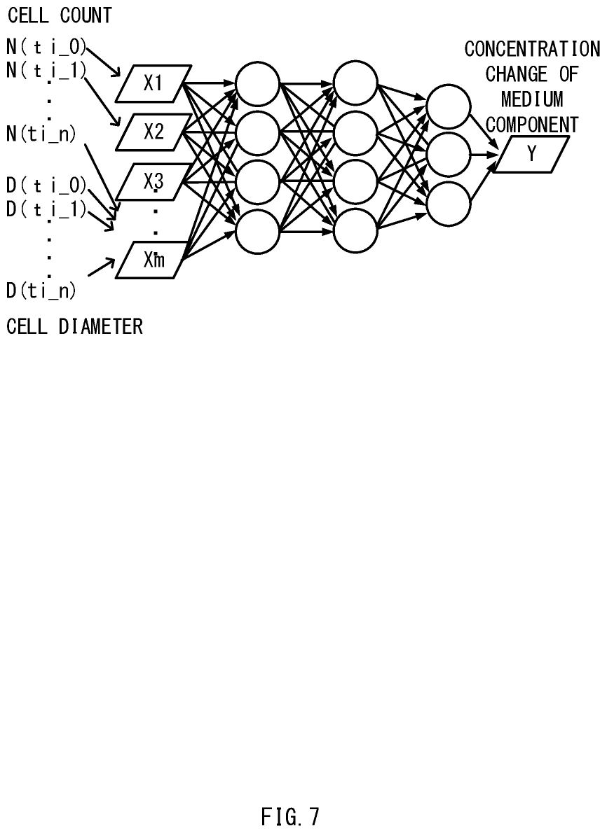

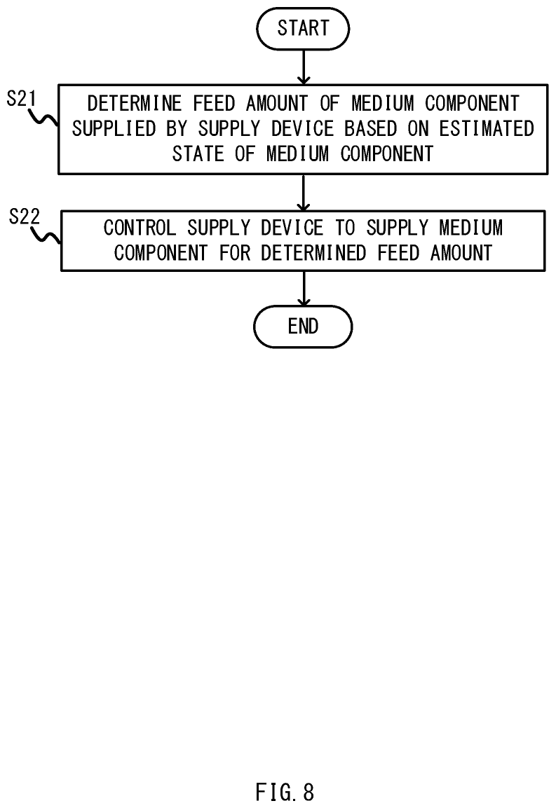

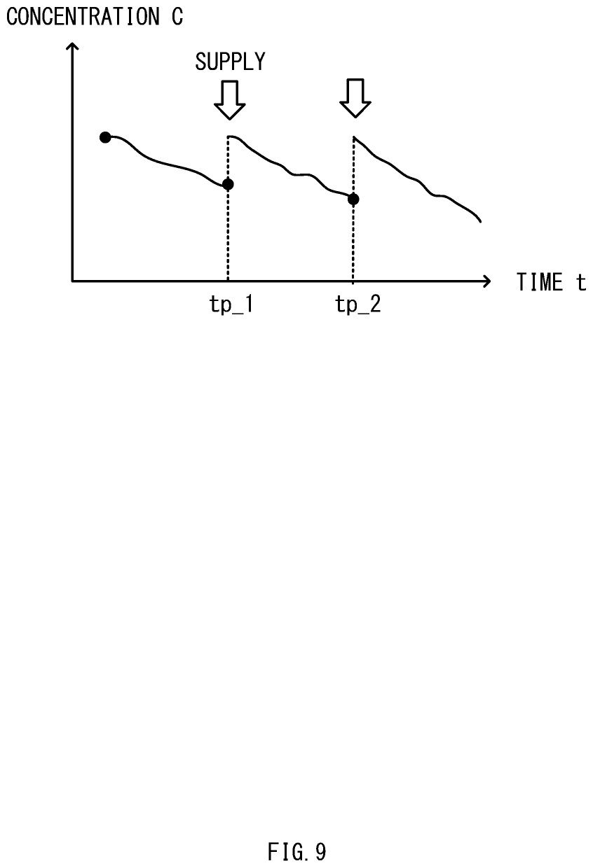

FIG. 4 is a flowchart illustrating an example of a process performed by the culture management apparatus 1 according to the present embodiment. FIG. 5 is a flowchart illustrating an example of a state estimation process. FIG. 6 is a diagram illustrating the cell change information. FIG. 7 is a diagram illustrating the learned model. FIG. 8 is a flowchart illustrating an example of a culture environment control process. FIG. 9 is a diagram illustrating an example of a concentration change of the specific medium component. Hereinafter, an estimation method in culture management using the culture management apparatus 1 will be specifically described with reference to FIGS. 4 to 9.

For example, the process illustrated in FIG. 4 is started according to an instruction of the user. The processor 11 of the control device 10 executing a program stored in the storage device 12 during the cell culture period.

When the process shown in FIG. 4 starts, the culture management apparatus 1 repeatedly photographs the inside of the culture vessel 110 (step S1). In step S1, the control device 10 controls the imaging device 120, so that the imaging device 120 repeatedly photographs the inside of the culture vessel 110 and acquires images. The images include the culture medium and the cells stored in the culture vessel 110.

Next, the culture management apparatus 1 performs the state estimation process of estimating the state of the culture medium (step S2). In step S2, the control device 10 performs to estimate the state of the specific medium component constituting the culture medium based on the images acquired in step S1. For example, the state estimation process illustrated in FIG. 5.

In the state estimation process illustrated in FIG. 5, the control device 10 generates information indicating a change that has occurred in at least one of a cell and a cell mass in the culture vessel 110. The information are generated based on the images acquired in step S1 (step S11). And then the control device 10 estimates the state of the specific medium component based on the information indicating the change (step S12). Note that the information indicating the change that has occurred in at least one of the cell and the cell mass generated from images in step S11 is hereinafter referred to as a cell change information.

In step S11, the control device 10 detects the cell change information by comparing images taken at different times. More specifically, for example, the control device 10 may perform object detection on each image using the learned model. And estimate a cell count, a cell diameter, and the like on the basis of the result of the object detection. The learned model is learned in advance by supervised learning regarding the form of the cell to be culture. For example, a deep learning convolutional neural network (CNN) may be used as the learned model. Furthermore, the control device 10 may generate the cell change information by arranging the cell count (or the number of cell masses), the cell diameter (cell mass diameter), and the like estimated for each image in order of image acquisition times. FIG. 6 illustrates the cell change information generated on the basis of the images. In this example, the cell change information includes information of a change in a cell count N and information indicating a change in a cell diameter D.

The cell change information is not limited to the change in the cell count and the change in the cell diameter, but desirably includes at least one of the change in the cell count and the change in the cell size. It is expected that the changes with respect to the cell count and cell size are causally related to an increase or decrease in components related to the cell metabolism. Therefore, by estimating the state of the specific medium component based on the cell change information including these the cell count and cell size changes, it is possible to perform estimation with high reliability. The change related to the cell count may be a change in the cell count itself, a change in a change rate (increase rate or decrease rate) of the cell count, or the like. In addition, the change related to the cell size may be a change in the cell diameter, a change in the change rate of the cell diameter, a change in the cell area (volume), a change in the change rate of the cell area (volume), or the like. The cell change information indicated by the cell change information may include, for example, a change in a shape of the cell or the cell mass, specifically, a change in roundness or sphericity. This is because when the cells constituting the cell mass die, its shape tends to collapse due to weakened adhesion between the cells. Therefore, it can be estimated that the cell mass is in a better state when the shape of the cell mass is closer to a true sphere (or a true circle).

In step S12, the control device 10 estimates the state of the specific medium component based on the cell change information generated in step S11, for example, using the learned model stored in the storage device 12. The learned model used in step S12 is a model in which a change that has occurred in the specific medium component with respect to a cell change information is learned by supervised learning. For example, a deep learning convolutional neural network (CNN) can be used. More specifically, for example, as shown in FIG. 7, the model may be a model in which the concentration change of the specific medium component with respect to the change in one or both of the cell count and the change in the cell diameter is learned.

The learned model illustrated in FIG. 7 is a model that input the cell count N and the cell diameter D to an input layer, and output the concentration change of the specific medium component from an output layer. The cell count N and the cell diameter D are included in the cell change information in each image acquisition time (ti_0 to ti_n). Using the learned model illustrated in FIG. 7, the control device 10 may estimate one or more of the concentration change and the concentration of the specific medium component, based on the concentration change obtained from the learned model and the initial information (e.g., concentration of the specific medium component in the initial medium).

Note that, in step S12, a model for estimating a change in the amount of the specific medium component may be used as the change that has occurred in the specific medium component. It is desirable to use a model that estimates at least one of the change in concentration and the change in amount of the specific medium component as the change that has occurred in the specific medium component. As a result, the feed amount to be supplied from the supply device 130 to the culture vessel 110 can be easily calculated from the estimation result.

Upon completion of the state estimation process in step S2, the culture management apparatus 1 performs the culture environment control process for controlling the culture environment in the culture vessel 110 (step S3). In step S3, the control device 10 controls the culture apparatus 100 based on the state of the specific medium component estimated in step S2, for example, by performing the culture environment control process illustrated in FIG. 8.

In the culture environment control process illustrated in FIG. 8, the control device 10 first determines the feed amount of the specific medium component supplied by the supply device 130 based on the state of the specific medium component estimated in step S2 (step S21), and then controls the supply device 130 to supply the specific medium component to the culture vessel 110 for the determined feed amount (step S22).

In step S21, for example, when the concentration of the specific medium component has decreased, the control device 10 calculates and determines an amount of the specific medium component necessary for increasing the concentration of the specific medium component from the reduced concentration to the concentration in the initial medium (hereinafter referred to as an initial concentration). In step S22, the control device 10 controls the supply device 130 such that the supply device 130 supplies the specific medium component to the culture vessel 110 for the calculated amount. the target concentration is not limited to the initial concentration. In step S21, the control device 10 only needs to supply the specific medium component so that the concentration of the specific medium component becomes an appropriate concentration. And the appropriate concentration only needs to be determined in advance according to the culture time (culture stage) and other factors.

In the process illustrated in FIG. 4 described above, the culture management apparatus 1 estimates the state of the medium component based on at least one of image. Therefore, it is possible to identify the state of the medium component without delay as compared with a conventional case where the culture medium is sampled and component analysis is performed by an analyzer. Therefore, by periodically repeating the process illustrated in FIG. 4 at an appropriate cycle, for example, the culture management apparatus 1 can suppress a significant variation in the state of the specific medium component as illustrated in FIG. 9. FIG. 9 illustrates an example in which a significant variation in the concentration of the specific medium component is suppressed. More specifically, FIG. 9 illustrates a state in which the supply device 130 supplies the specific medium component to the culture vessel 110 at time tp_1 and time tp_2, and as a result, the concentration of the specific medium component is recovered to an appropriate concentration (in this example, the initial concentration) at time tp_1 and time tp_2. FIG. 9 assumes a case in which the state (concentration) at tp_1 is estimated by the image acquired at tp_1, and the specific medium component is further supplied at tp_1. There may be a time difference between an image acquisition timing and a feed timing. For example, there is a time difference therebetween, and the supply may be started after a certain time elapses from the image acquisition. In addition, the culture management apparatus 1 automatically adjusts the culture environment according to the estimated state. Therefore, it is possible to maintain the quality of cells by supplying an appropriate medium component while reducing a burden on the user who manages the culture environment. Furthermore, in the culture management apparatus 1, the analysis is performed based on the determination by image processing without expensive and many type of measurement instruments. Therefore, the introduction cost can also be suppressed.

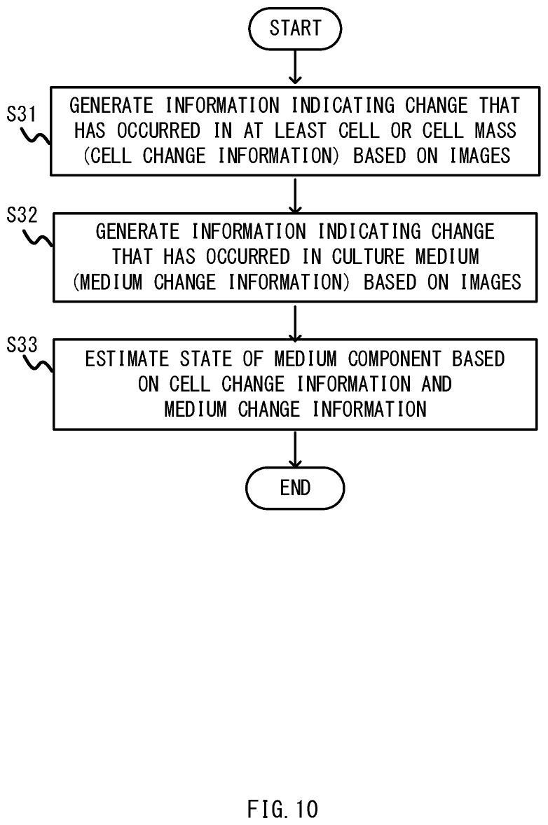

FIG. 10 is a flowchart illustrating another example of the state estimation process. The above-described embodiment gives an example of estimating the state of a specific medium component based on the cell change information indicating a change in the cell count, the cell size, the cell shape, and the like. The culture management apparatus 1 may perform the state estimation process illustrated in FIG. 10 instead of the state estimation process illustrated in FIG. 5.

Also in the state estimation process illustrated in FIG. 10, the control device 10 generates the cell change information based on the images acquired in step S1 (step S31). The process in step S31 is similar to the process in step S11 in FIG. 5.

The control device 10 further generates information indicating a change that has occurred in the culture medium in the culture vessel 110 based on the images acquired in step S1 (step S32). Note that the processes in step S31 and step S32 may be performed prior to the process in step S33. In other words, the process in step S32 may be performed before the process in step S31, and the process in step S31 and the process in step S32 may be executed in parallel in terms of time. Thereafter, the control device 10 estimates the state of the specific medium component based on the cell change information generated in step S31 and the change information generated in step S32 (step S33). The information indicating the change that has occurred in the culture medium generated from images in step S32 is hereinafter referred to as a medium change information. In addition, the process in step S33 is similar to that in step S12 in FIG. 5 except that the state of the specific medium component is estimated using the learned model in which the cell change information and the change that has occurred in the specific medium component with respect to the medium change information are learned by supervised learning.

In step S32, the control device 10 detects the medium change information by comparing images captured at different times. More specifically, for example, the control device 10 detects a change in the color of the culture medium by comparing images. The medium change information to be detected includes impurities in the culture medium, transparency of the culture medium, and the like. Thus, it is preferable that the change includes the color of the culture medium but is not limited to the color of the culture medium.

When the cell metabolism proceeds, the metabolic product increases to change the pH of the culture medium from neutral to acidic. Then, the color of the culture medium changes from red to yellow due to an action of phenol red which is a pH indicator contained in the culture medium. Thus, it is expected that the change in the color of the culture medium is causally related to an increase or decrease in components related to the cell metabolism. Instead of the state estimation process illustrated in FIG. 5, the culture management apparatus 1 performs the state estimation process illustrated in FIG. 10. The culture management apparatus 1 estimate the state of the specific medium component on the basis of the medium change information including the change in the color of the culture medium and the cell change information, thereby enabling estimation with higher reliability.

In addition, it is possible to detect a sudden change in the color of the culture medium to yellow caused by contamination of microorganisms. Therefore, necessary measures such as suspension of culturing can be performed at an early stage.

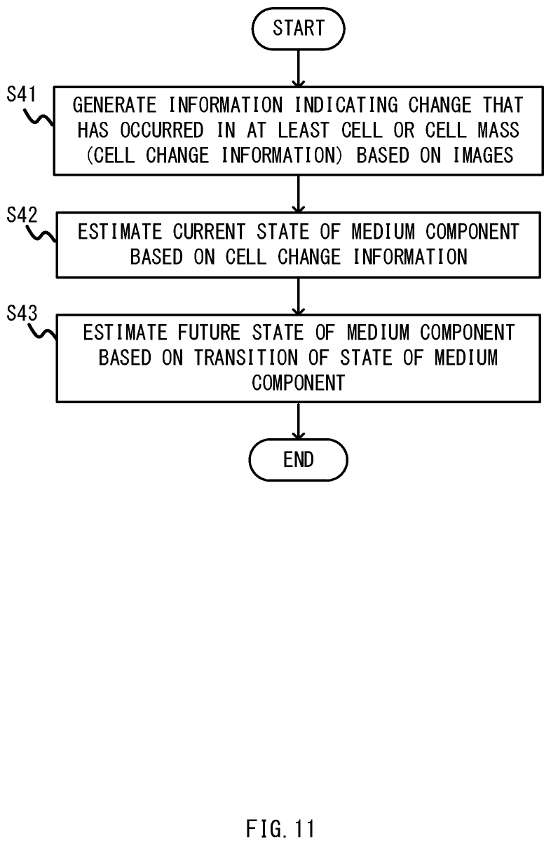

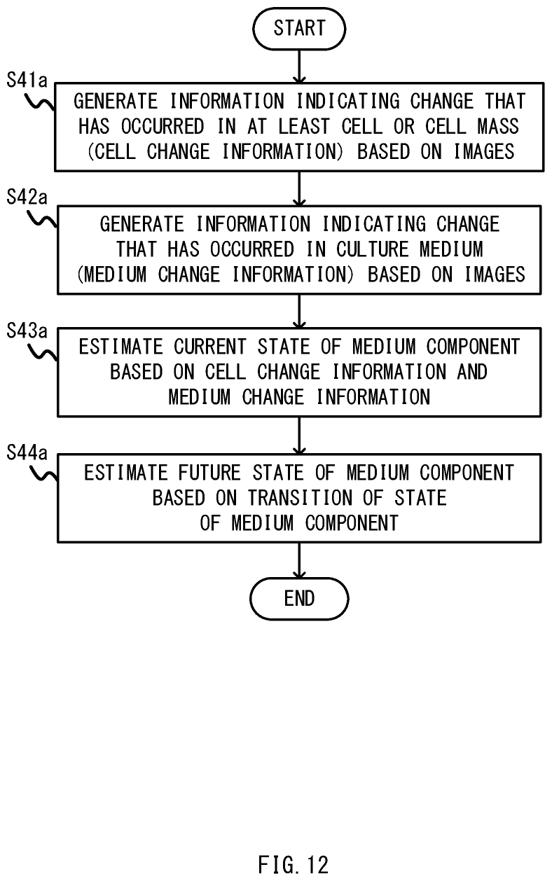

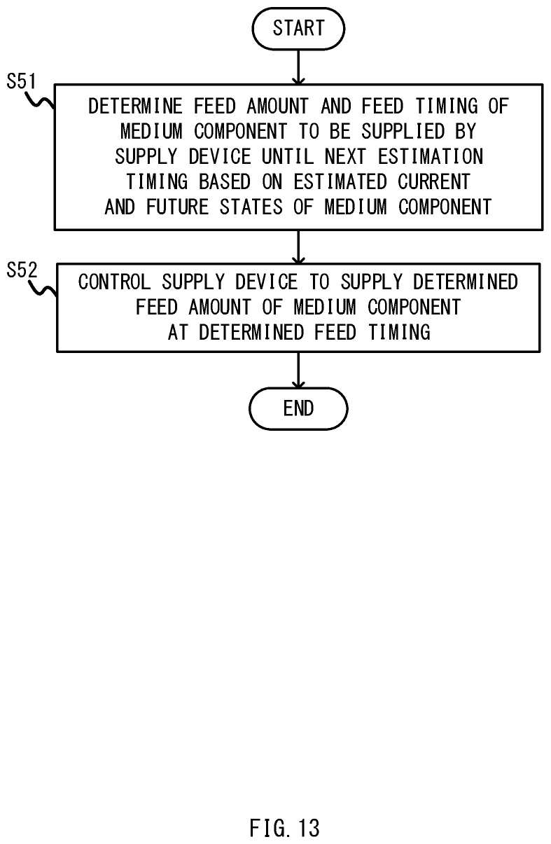

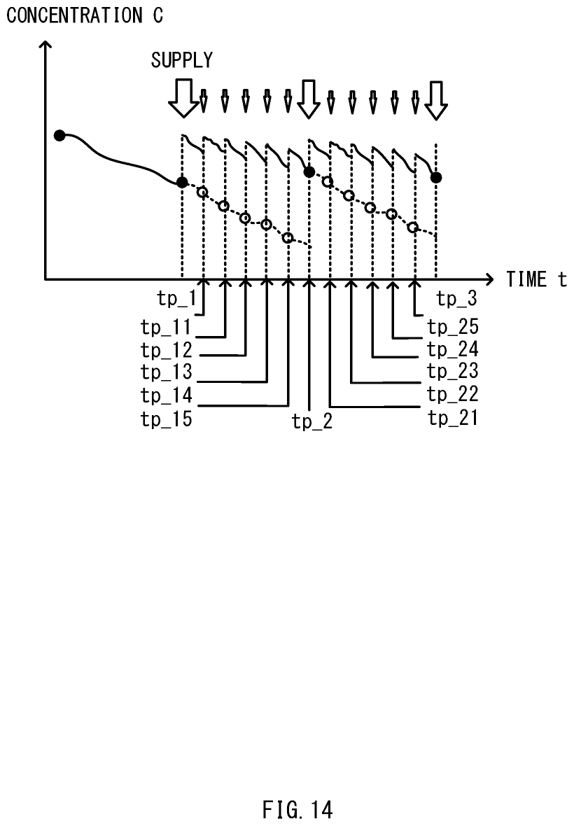

FIGS. 11 and 12 are flowcharts illustrating still another example of the state estimation process. FIG. 13 is a flowchart illustrating another example of the culture environment control process. FIG. 14 is a diagram illustrating another example of the concentration change of the specific medium component. The embodiment described above gives the example of estimating the state of the medium component at each of the image acquisition time. In addition to the state of the medium component at the time of photographing, a future state of the medium component may be estimated. Hereinafter, an example will be described in which the state estimation process illustrated in FIG. 11 or 12 is performed for estimating the current and future states of the medium components, instead of the state estimation process illustrated in FIG. 5. And the culture environment control process illustrated in FIG. 13 is performed for supplying the medium component a plurality of times during a period between the current estimation timing and the next estimation timing, instead of the culture environment control illustrated in FIG. 8.

In the state estimation process illustrated in FIG. 11, the control device 10 generates the cell change information based on images acquired in step S1 (step S41). Then, the control device 10 estimates the current state of the specific medium component based on the cell change information (step S42). The processes in step S41 and step S42 are similar to the processes in step S11 and step S12 in FIG. 5.

The control device 10 further estimates the future state of the specific medium component based on the transition of the state of the specific medium component (step S43). In step S43, the control device 10 estimates the future state of the specific medium component using a learned model different from the learned model used in step S42. For example, the learned model is inputted one or more of the current state of the specific medium component and the past state of the specific medium component at each time estimated in step S42 performed so far The learned model used in step S43 is, for example, a model obtained by learning time series data of the state of the specific medium component by supervised learning. For example, a deep learning recurrent neural network (RNN) or a long short-time memory (LSTM) that is an extension of the RNN can be used. In other words, in step S42 and step S43, the current and future medium states are estimated from the cell change information and the medium change information. Note that an example is illustrated the current and future medium states are estimated based on the cell change information in FIG. 11. As illustrated in FIG. 12, the current and future medium states may be estimated based on both the cell change information and the medium change information. In this case, the control device 10 may first generate the cell change information and the medium change information from the images, similarly to the processes in step S31 and step S32 in FIG. 10 (step S41a and step S42a). Then, the control device 10 may estimate the current and future states of the medium components based on the cell change information and the medium change information (step S43a and step S44a).

In the culture environment control process is illustrated in FIG. 13. The control device 10 determines the feed amount and the feed timing of the specific medium component to be supplied by the supply device 130 until the next estimation timing based on the current and future states of the specific medium component estimated in step S42 and step S43 (step S51). Then, the control device 10 controls the supply device 130 to supply the feed amount determined of the specific medium component to the culture vessel 110 at the feed timing determined in step S51 (step S52).

In step S51, for example, the control device 10 may match the feed timing with the timing of the state estimated in step S42 and step S43. Specifically, for example, when the state (concentration) at the current time tp_1 is estimated in step S42, the control device 10 may determine the feed timing as the current time tp_1 in step S51. And when the states (concentrations) at future times tp_11, tp_12, and so on to tp_15 after the current time tp_1 are estimated in step S43, the control device 10 may determine the feed timing as the future times tp_11, tp_12, and so on to tp_15 in step S51. Note that a black circle indicated in FIG. 14 is an estimation result at the current time, and a white circle indicated in FIG. 14 is an estimation result at a future time. In other words, in the example illustrated in FIG. 14, at the time tp_1, a state at the current time tp_1 is estimated and states at future times tp_11, tp_12, tp_13, tp_14, and tp_15 are also estimated. In step S51, the control device 10 may determine the feed amount at each feed timing so that the estimated concentration at each feed timing is adjusted to the concentration (initial concentration) in the initial medium. In the example illustrated in FIG. 14, at time tp_1, time tp_11, time tp_12, time tp_13, time tp_14, and time tp_15, the specific medium component is supplied for the feed amount determined for each time (timing) at time tp_1, and as a result, a change in concentration indicated by a solid line occurs in the culture vessel 110. On the other hand, a dotted line illustrated in FIG. 14 indicates a change in concentration estimated in the culture vessel 110 when the feed control is not executed. More specifically, the dotted line from time tp_1 to time tp_2 indicates the concentration change when state estimation is not performed and supply control is not executed at time tp_1. Further, the dotted line from time tp_2 to time tp_3 indicates the concentration change when state estimation is not performed and supply control is not executed at time tp_2. Note that FIG. 14 shows an example in which the medium component is supplied with the initial concentration as a target, but the target concentration is not limited to the initial concentration. In step S51, the control device 10 only needs to feed the specific medium component to achieve the appropriate concentration of the specific medium component, and the appropriate concentration only needs to be determined in advance according to the culture time (culture stage) and other factors. Note that the control device 10 may estimate the amount, instead of the concentration of the specific medium component. In this case, the specific medium component may be supplied so that the amount of the specific medium component becomes an appropriate amount. Furthermore, the example illustrated in FIG. 14 also illustrates that, at the time tp_2, a state at the current time tp_2 is estimated and states at future times tp_21, tp_22, tp_23, tp_24, and tp_25 are estimated. Furthermore, the example illustrated in FIG. 14 also illustrates that a state at the current time tp_3 is estimated at the time tp_3.

As described above, by estimating the change in the state of the specific medium component during the period between the processes illustrated in FIG. 4 that are periodically performed and supplying the medium component a plurality of times during the period, the culture management apparatus 1 can further suppress the change in the state of the specific medium component as illustrated in FIG. 14.

FIG. 9 illustrates an example in which the timing of image acquisition coincides with the feed timing, and FIG. 14 illustrates an example in which the timing of image acquisition differs from the feed timing. However, either of these controls may be performed. As illustrated in FIG. 9, even when the image acquisition timing and the feed timing coincide, it is possible to avoid a large deviation of the concentration of the specific medium component from the appropriate state by sufficiently shortening the image acquisition interval. In addition, improvement in estimation accuracy can also be expected by using images acquired at short sampling intervals. On the other hand, as illustrated in FIG. 13, when the timing of image acquisition and the feed timing differ from each other, by setting a feed frequency to be higher than a frequency of image acquisition, it is possible to obtain a similar effect to that when the image acquisition interval is substantially shortened. Therefore, it is possible to prevent the concentration of the specific medium component from greatly deviating from the appropriate state.

Note that FIG. 11 illustrates an example of estimating the state of the specific medium component in the future from the time-series data of the state of the specific medium component. However, the cell change information that is the time-series data over the future may be estimated from the cell change information that is the time-series data up to the present, and the current and future states of the medium component may be estimated based on the estimated cell change information over the future.

### Second Embodiment

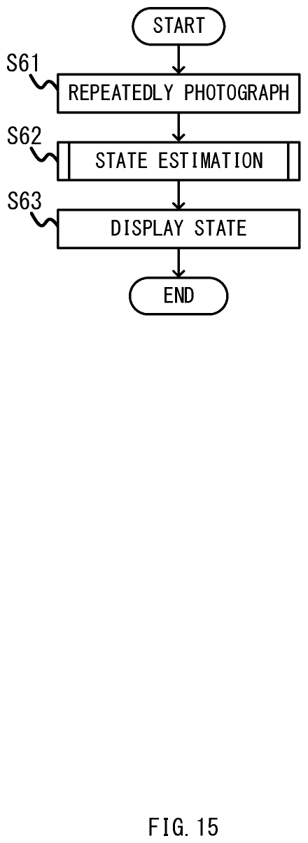

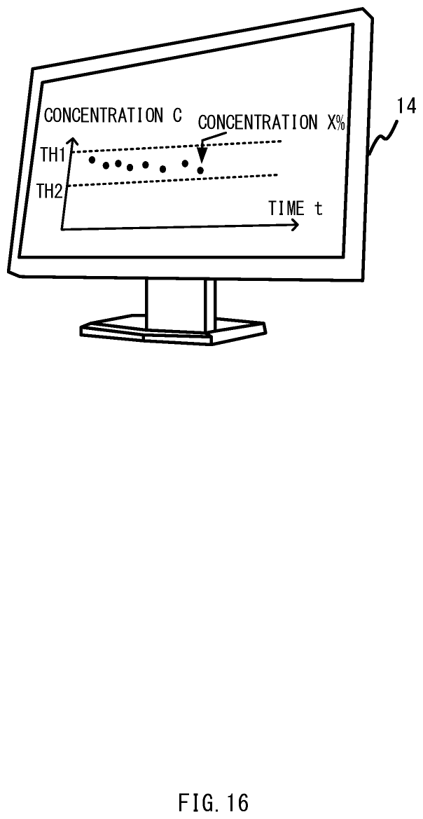

FIG. 15 is a flowchart illustrating an example of a process performed by a culture management apparatus according to the present embodiment. FIG. 16 is a diagram illustrating an example of a screen displayed on a display device. Hereinafter, a specific example of an estimation method in culture management using the culture management apparatus according to the present embodiment will be described with reference to FIGS. 15 and 16. A configuration of the culture management apparatus according to the present embodiment may be, for example, the same as that of the culture management apparatus 1 according to the first embodiment. The culture management apparatus according to the present embodiment may be different from the culture management apparatus 1 only in that the process illustrated in FIG. 15 is performed instead of the process illustrated in FIG. 4. However, the culture management apparatus according to the present embodiment may be applied not only to fed-batch culture but also to continuous culture or batch culture.

When the process shown in FIG. 15 starts, the culture management apparatus 1 repeatedly photographs an inside of the culture vessel 110 (step S61), and performs a state estimation process of estimating the state of the culture medium on the basis of a plurality of photographed images (step S62). The process in step S61 and step S62 is similar to the process in step S1 and step S2 in FIG. 4.

When the state of a specific medium component is estimated by the state estimation process, the culture management apparatus according to the present embodiment displays the estimated state instead of automatically adjusting the culture environment based on the estimated state (step S63).

In step S63, for example, the control device 10 may cause the display device 14 of the control device 10 to display the estimated state of the specific medium component. The state displayed on the display device 14 may be only the latest state, or may be both the latest state (concentration) and the past state (concentration) as illustrated in FIG. 16. Note that, for example, the control device 10 may notify the user terminal 2 of the estimated state of the specific medium component, and cause the display device of the user terminal 2 to display the estimated state of the specific medium component. The state of the specific medium component to be displayed is not limited to the concentration of the specific medium component, and may be an amount of the specific medium component. In addition, both the concentration and the amount may be displayed, and these may be displayed on a graph using different colors or marks. Furthermore, in addition to the state of the specific medium component (concentration, amount), a total amount of supplied medium component and the like may be displayed.

Also in the culture management apparatus according to the present embodiment, the state of the medium component is estimated based on an image. Therefore, similarly to the culture management apparatus 1 according to the first embodiment, it is possible to identify the state of the medium component without delay. Therefore, it is possible to suppress a significant variation in the state of the specific medium component. The culture management apparatus according to the present embodiment notifies the user of the state by displaying the estimation result on the display device, instead of automatically adjusting the culture environment based on the estimation result. Therefore, the user can recognize a change in the state at an early stage and take an appropriate measure such as addition of a feed medium. Also in the present embodiment, as in the first embodiment, a future state of the medium component may be estimated in addition to the state of the medium component at the time of photographing. In this case, the state displayed on the display device 14 may include at least one of the latest state (concentration), the past state (concentration), and the future state (concentration), or may include all of these states. By notifying the user of the predicted future state, the user can deal with the problem with a time margin, so that the user can take a more appropriate action.

### Third Embodiment

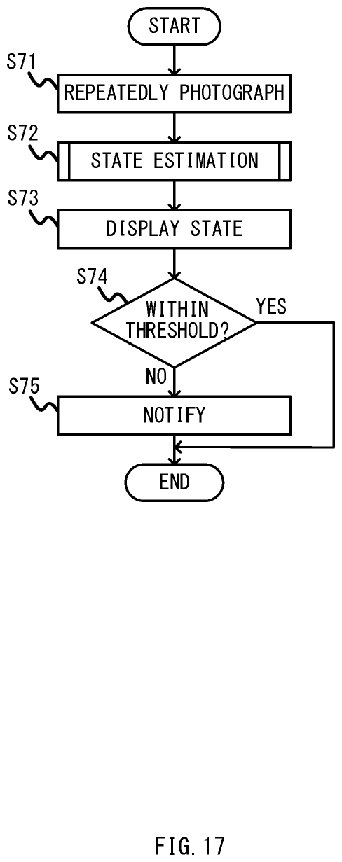

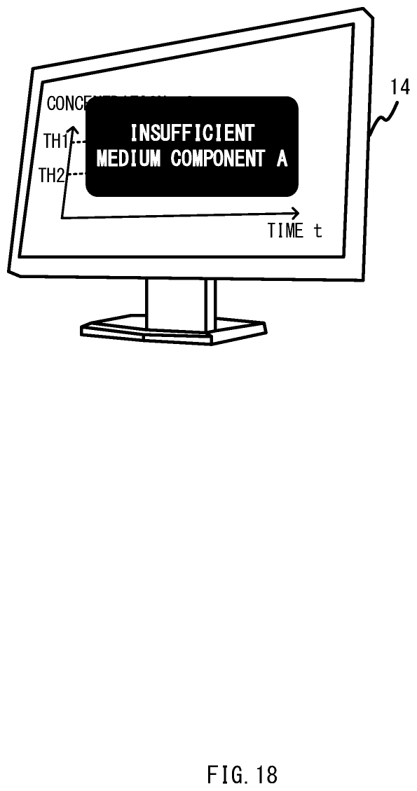

FIG. 17 is a flowchart illustrating an example of a process performed by a culture management apparatus according to the present embodiment. FIG. 18 is a diagram illustrating another example of a screen displayed on a display device. Hereinafter, a specific example of an estimation method in culture management using the culture management apparatus according to the present embodiment will be described with reference to FIGS. 17 and 18. Note that a configuration of the culture management apparatus according to the present embodiment may be the same as, for example, the culture management apparatus 1 according to the first embodiment and the culture management apparatus according to the second embodiment. The culture management apparatus according to the present embodiment may be different from the culture management apparatus according to the second embodiment only in that a process illustrated in FIG. 17 is performed instead of the process illustrated in FIG. 15.

When the process illustrated in FIG. 17 starts, the culture management apparatus 1 repeatedly photographs an inside of the culture vessel 110 (step S71), performs a state estimation process of estimating the state of the culture medium based on a plurality of photographed images (step S72), and displays an estimated state (step S73). Processes in step S71 to step S73 is similar to the processes in step S61 to step S63 in FIG. 15.

In the culture management apparatus according to the present embodiment, after the display device 14 displays the estimated state, and the control device 10 determines whether or not a numerical value (e.g., concentration value) is within a predetermined threshold range (step S74). The numerical value indicates the estimated state of the specific medium component. Then, when the estimated numerical value is out of the predetermined threshold range, the control device 10 notifies the user of the culture management apparatus 1 (step S75). Note that FIG. 18 is an example of notifying the user by displaying information on the display device 14, but the notification to the user is not limited to the display of the information on the display device 14. For example, the notification may be given to the user using voice, vibration, light, or the like, or may be given to the user by, for example, transmitting an e-mail to the user terminal 2.

Also in the culture management apparatus according to the present embodiment, same effects as those of the culture management apparatus according to the second embodiment can be obtained. In addition, in the culture management apparatus according to the present embodiment, when the state of the specific medium component falls outside the threshold range, notification of the state is given to the user, so that it is possible to avoid the user from overlooking a significant change in the state of the specific medium component.

### Fourth Embodiment

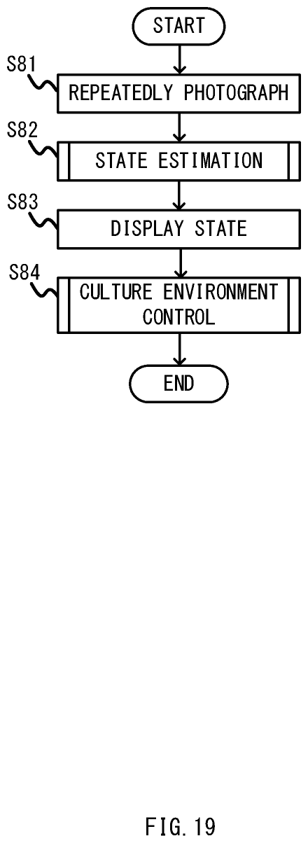

FIG. 19 is a flowchart illustrating an example of a process performed by a culture management apparatus according to the present embodiment. A configuration of the culture management apparatus according to the present embodiment may be, for example, the same as that of the culture management apparatuses according to the first to third embodiments. The culture management apparatus according to the present embodiment may be different from the culture management apparatus according to the second embodiment only in that a process illustrated in FIG. 19 is performed instead of the process illustrated in FIG. 15.

In the process illustrated in FIG. 19, the culture management apparatus repeatedly photographs an inside of the culture vessel 110 (step S81), and performs a state estimation process of estimating the state of the culture medium based on a plurality of photographed images (step S82). Then, in addition to displaying the estimated state (step S83), the culture management apparatus performs a culture environment control process in step S84. The displaying step and the culture environment control step are different from the culture management apparatus according to the second embodiment. The culture environment control process in step S84 may be the culture environment control process illustrated in FIG. 8 or the culture environment control process illustrated in FIG. 13. In addition, an order of the processes in step S83 and step S84 is not limited to this example. The process in step S83 may be performed after the process in step S84, or step S83 and step S84 may be performed in parallel in terms of time. When the control of the culture environment is automatically started, a state of the medium component changes from the time of estimation. Therefore, in step S83, it is desirable that the information on the state to be displayed is displayed in a way such that it can be understood that it is the state at the time of estimation.

Also in the culture management apparatus according to the present embodiment, effects obtained by the culture management apparatus according to the first embodiment and the effect obtained by the culture management apparatus according to the second embodiment can be simultaneously obtained. Furthermore, the culture management apparatus according to the present embodiment may have a notification function similarly to the culture management apparatus according to the third embodiment.

### Fifth Embodiment

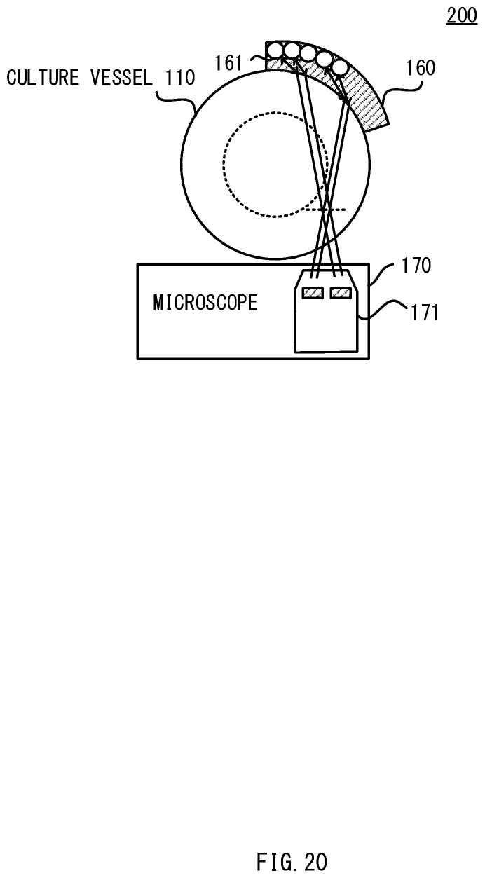

FIG. 20 is a diagram illustrating a configuration of a culture apparatus 200 according to the present embodiment. Hereinafter, the configuration of the culture apparatus 200 according to the present embodiment will be described with reference to FIG. 20. A culture management apparatus according to the present embodiment is different from the culture management apparatus 1 according to the first embodiment in that the culture apparatus 200 illustrated in FIG. 20 is provided instead of the culture apparatus 100. Although not illustrated, the culture management apparatus according to the present embodiment is similar to the culture management apparatus 1 in that the control device 10 is included. The culture management apparatus according to the present embodiment may perform any of the process illustrated in FIG. 4, the process illustrated in FIG. 15, the process illustrated in FIG. 17, and the process illustrated in FIG. 19.

As illustrated in FIG. 20, the culture apparatus 200 is different from the culture apparatus 100 in that a microscope 170, and that a retroreflective member 160 attached to the culture vessel 110 is provided. The microscope 170 is an example of an imaging device instead of the imaging device 120. Similarly to the imaging device 120, the microscope 170 is a device that photographs an inside of the culture vessel 110 from an outside of the culture vessel 110 to capture an image including a liquid culture medium and cells suspended in the liquid culture medium. In the microscope 170, for example, a distal end of the objective lens 171 is arranged to face a side surface of the culture vessel 110. The microscope 170 can also repeatedly photograph the inside of the culture vessel 110.

Note that the microscope 170 is a phase contrast microscope that photographs an object by a phase contrast observation method. However, the microscope 170 is not limited to the phase contrast microscope, and may photograph an object by, for example, a bright field observation method.

The retroreflective member 160 is used to cancel a lens effect received on the side surface of the culture vessel 110 by a light emitted from the microscope 170. The retroreflective member 160 has an array in which a large number of minute reflective elements 161 are arranged in the horizontal direction. The reflective element 161 is, for example, a prism or a spherical glass bead. The retroreflective member 160 reflects an incident light by the reflective element 161 and causes the reflected light to travel in the same optical path as that at the time of incidence in the opposite direction.

When the inside of the culture vessel 110 is photographed from the outside of the culture vessel 110, an image quality generally depends on a shape of the culture vessel 110. However, by using the microscope 170 together with the retroreflective member 160, the lens effect generated on the side surface of the culture vessel 110 can be canceled, as described, for example, in WO 2019/163167A. Therefore, the inside of the culture vessel 110 can be photographed with stable performance from the outside of the culture vessel 110.

Also by the culture management apparatus according to the present embodiment, by executing any one of the process illustrated in FIG. 4, the process illustrated in FIG. 15, the process illustrated in FIG. 17, and the process illustrated in FIG. 19, the same effects as those of the culture management apparatuses according to the first to fourth embodiments can be obtained.

### Sixth Embodiment

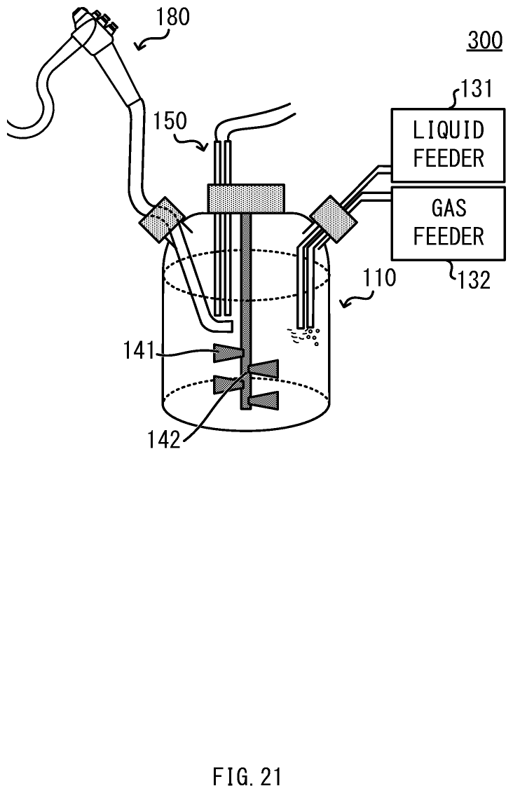

FIG. 21 is a diagram illustrating a configuration of a culture apparatus 300 according to the present embodiment. Hereinafter, a configuration of the culture apparatus 300 according to the present embodiment will be described with reference to FIG. 21. A culture management apparatus according to the present embodiment is different from the culture management apparatus 1 according to the first embodiment in that the culture apparatus 300 illustrated in FIG. 21 is provided instead of the culture apparatus 100. Although not illustrated, the culture management apparatus according to the present embodiment is similar to the culture management apparatus 1 in that the control device 10 is included. The culture management apparatus according to the present embodiment may perform any of the process illustrated in FIG. 4, the process illustrated in FIG. 15, the process illustrated in FIG. 17, and the process illustrated in FIG. 19.

As illustrated in FIG. 21, the culture apparatus 300 is different from the culture apparatus 100 in that an endoscope 180, which is an example of an imaging device, is provided instead of the imaging device 120. Unlike the imaging device 120 and the microscope 170, the endoscope 180 is a device that photographs an inside of the culture vessel 110 from the inside of the culture vessel 110 to capture an image including a liquid culture medium and cells suspended in the liquid culture medium. The culture apparatus 300 may further include a video processor or a lighting apparatus (not illustrated) connected to the endoscope 180.

Also in the culture management apparatus according to the present embodiment, by executing any one of the process illustrated in FIG. 4, the process illustrated in FIG. 15, the process illustrated in FIG. 17, and the process illustrated in FIG. 19, same effects as those of the culture management apparatuses according to the first to fifth embodiments can be obtained. In addition, in the endoscope 180, since photographing is performed from the inside of the culture vessel 110, the inside of the culture vessel 110 can be photographed with stable performance regardless of the shape of the culture vessel 110.

The above-described embodiments illustrate specific examples in order to facilitate understanding of the invention. However, the present invention is not limited to these embodiments. Variations obtained by modifying the above-described embodiments and alternatives to the above-described embodiments can be included. More specifically, in each embodiment, the components can be modified without departing from the spirit and scope thereof. In addition, a new embodiment can be implemented by appropriately combining a plurality of components disclosed in one or more embodiments. In addition, some components may be deleted from the components illustrated in the embodiments, or some components may be added to the components illustrated in the embodiments. Furthermore, process procedures described in each embodiment may be performed in a different order as long as there is no contradiction. In other words, the culture management apparatus, the estimation method, and the computer-readable medium of the present invention can be variously modified and changed without departing from the claims.

In the above-described embodiments, the examples focused on a change in cell count and cell size in order to estimate the state of the specific medium component in the culture medium. However, the state of the medium component to be controlled may be estimated on the basis of, for example, the number of divisions of cells per unit time, a ratio of cells during division, or the like, in addition to or instead of the above-described elements. When the state of the medium component to be controlled, which is related to cell metabolism, is good, it is assumed that, because nutrients are distributed to the cells, cell division becomes active and a proportion of dividing cells also increases. As a result, it is assumed that the number of divisions per hour increases. On the other hand, when the state of the medium component to be controlled is not good, because nutrients are poorly distributed to the cells, a frequency of division decreases, and the average time interval between divisions increases. As a result, it is assumed that the number of dividing cells also decreases. As described above, since the number of divisions and the dividing cell count are also expected to have a causal relationship with the increase or decrease of components related to cell metabolism, the state of the specific medium component may be estimated based on the cell change information including these changes.

In the embodiments described above, the state of the specific medium component is estimated based on the image, but the state of another medium component may be further estimated based on the estimated state of the specific medium component. For example, when a consumption amount of glucose is estimated on the basis of an image, a production amount of a metabolite, such as lactic acid, may be estimated using a cell metabolism model on the basis of an amount of glucose consumed or an amount of glucose newly supplied.

The above-described embodiments give the examples in which the change that appears in the image is mainly detected from the images, and the state of the medium component is estimated based on the change. However, the state of the medium component may be estimated based on each of the images. For example, the state of the specific medium component may be estimated based on information detected from one image at a certain time, such as the dividing cell count described above.

Repeated photographing performed in the above-described embodiments may be performed periodically or aperiodically. In a case where photographing is performed aperiodically, the change information calculated from the images may be converted into change information per unit time and used for estimating the state of the medium component.

The above-described embodiments give the examples of estimating the state using the predetermined learned model. However, the learned model used for the state estimation may be selected by the user from among a plurality of learned models, or may be selected by the control device 10 on the basis of information input by the user. Furthermore, in order to support the selection of the learned model by the user, information (e.g., cell type, other culture environments) used at the time of learning of each of the plurality of learned models may be displayed at the time of selecting the learned model.

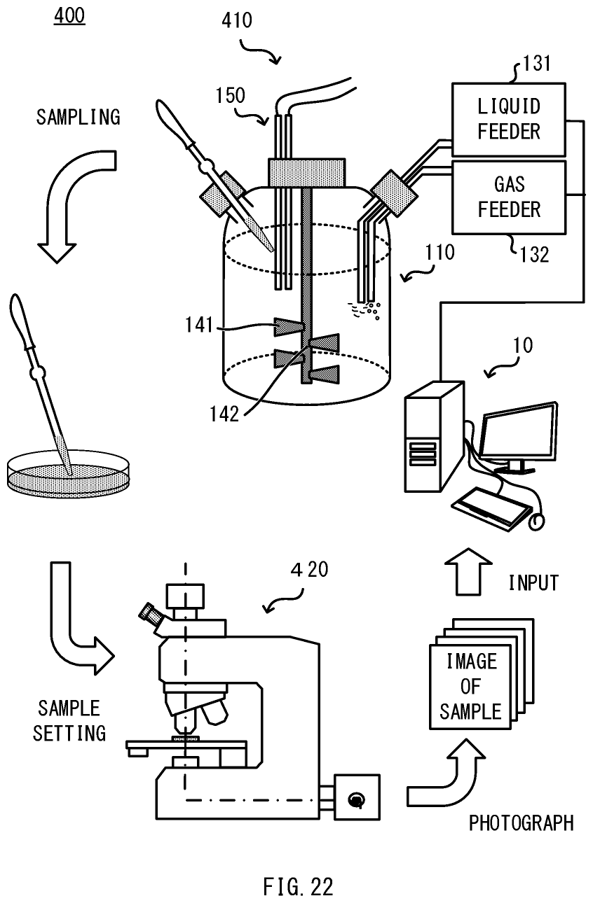

The above-described embodiments give the examples of estimating the state of the specific medium component based on the images obtained by photographing the culture medium and the cells in the culture vessel. However, each of the images may be an image obtained by photographing a sample including the culture medium and the cells sampled from the culture vessel, or may be an image of a sample sampled at different times. In this case, the culture management apparatus may be, for example, a culture management apparatus 400 including a microscope 420, as illustrated in FIG. 22, as the imaging device. The culture management apparatus 400 may include a culture apparatus 410 and the microscope 420. Note that the culture apparatus 410 is the same as the culture apparatus 100 except that the imaging device is not included. The control device 10 included in the culture apparatus 410 may estimate the state of the specific medium component in the culture medium based on an image captured by the microscope 420 provided outside the culture apparatus 410.

What is claimed is:

1. A culture management apparatus comprising:
a control device that estimates a state of a specific medium component constituting a culture medium based on images
wherein the images include both the culture medium and a cell in the culture medium acquired at different times.

2. The culture management apparatus according to claim 1,
wherein the control device
generates cell change information indicating a change that has occurred in the cell in the culture medium based on the images, and
estimates the state of the specific medium component based on the cell change information.

3. The culture management apparatus according to claim 2,
wherein the cell change information includes at least one of a change of a number of the cell and a change of a cell size.

4. The culture management apparatus according to claim 2,
Wherein the control device
detects an object included in each of the images, and
generates the cell change information based on a result of the detected object.

5. The culture management apparatus according to claim 1,
wherein the control device
generates medium change information indicating a change that has occurred in the culture medium based on the images, and
estimates the state of the specific medium component based on the cell change information and the medium change information.

6. The culture management apparatus according to claim 5,
wherein the medium change information includes a change of color in the culture medium.

7. The culture management apparatus according to claim 2 further comprising,
a memory device stores a learned model learned by supervised learning,
wherein the learned model learns specific medium component change information with respect to at least one of the cell change information and medium change information,
wherein the specific medium component change information indicates a change has occurred in the specific medium component,
wherein the medium change information indicates a change that has occurred in the culture medium, and
wherein the control device estimates the state of the specific medium component using the learned model.

8. The culture management apparatus according to claim 7,
wherein the specific medium component change information includes at least one of a change in concentration of the specific medium component and a change in amount of the specific medium component.

9. The culture management apparatus according to claim 1, further comprising
a culture apparatus including a culture vessel housing the culture medium and the cell,
wherein the control device controls the culture apparatus based on the estimated state of the specific medium component.

10. The culture management apparatus according to claim 9,
wherein the culture apparatus includes a supply device that supplies a predetermined substance to the culture vessel, and
wherein the control device determines a feed amount of the predetermined substance supplied from the supply device to the culture vessel based on the estimated state of the medium component.

11. The culture management apparatus according to claim 10,
wherein the determined feed amount of the predetermined substance is configured to suppress a change in the state of the specific medium component.

12. The culture management apparatus according to claim 10,
wherein the predetermined substance includes the specific medium component.

13. The culture management apparatus according to claim 1, further comprising
a display device,
wherein the control device causes the display device to display the estimated state of the specific medium component.

14. The culture management apparatus according to claim 1,
wherein the control device compares the estimated state of the specific medium component and a predetermined threshold range,
wherein the control device notifies a user the comparison result.

15. The culture management apparatus according to claim 1, further comprising
an imaging device acquired the images of the culture medium and the cell in the culture medium.

16. The culture management apparatus according to claim 1,
wherein the cell includes at least one of the cell and a cell mass.

17. An estimation method comprising:
estimating a state of a specific medium component constituting a culture medium based on images including both the culture medium and a cell in the culture medium photographed at different times.

18. A non-transitory computer-readable medium storing a program causing a computer to implement:
estimating a state of a specific medium component constituting a culture medium based on images including both the culture medium and a cell in the culture medium photographed at different times.
